(19) United States
(12) Patent Application Publication
(10) Pub. No.: US 2025/0275817 A1
(43) Pub. Date: Sep. 4, 2025

(54) INTERFACE FOR CONNECTING A DRIVE UNIT TO A MEDICAL INSTRUMENT

(71) Applicant: KARL STORZ SE & Co. KG, Tuttlingen (DE)

(72) Inventors: Martin HOFFMANN, Berlin (DE); Torben KLINGELS, Landshut (DE)

(73) Assignee: KARL STORZ SE & Co. KG, Tuttlingen (DE)

(21) Appl. No.: 19/045,160

(22) Filed: Feb. 4, 2025

(30) Foreign Application Priority Data
Feb. 5, 2024 (DE) 102024103154.9

(51) Int. Cl. A61B 34/30; A61B 17/00

(52) U.S. Cl. CPC A61B 34/30; A61B 2017/00477

(57) ABSTRACT

The invention relates to an interface (10) for connecting a drive unit (12) to a medical instrument (14), comprising:
at least one drive-side connecting element (16) with at least one first coupling surface (18), and
at least one instrument-side connecting element (20) with at least one second coupling surface (22),
wherein the two connecting elements (16, 20) can be coupled to one another via the respective coupling surfaces (18, 22) with the interposition of a continuous medical barrier (24), and
at least one holding device (26) which is designed to hold the connecting elements (16, 20) together in a coupled state.

The present invention relates to an interface for connecting a drive unit to a medical instrument, in particular as part of a medical robot system.

Medical robot systems, especially in the form of conventional medical master-slave robot systems, which are used, for example, for the mechanical guidance of medical and/or surgical instruments and the control of end effectors, usually have a detachable connection at an interface between motor drives on the robot side and the surgical instruments. In this case, the drive unit and the surgical instrument are usually coupled to each other in a force-fitting manner via a gear arrangement.

In order to meet the requirements for a sterile treatment environment, a medical barrier comprising a plastics film is provided in the region of the interface, which plastics film separates a drive-side part of the interface from an instrument-side part of the interface. In order to transmit at least one driving force and/or at least one driving torque between the parts of the interface, known medical barriers have inserts and/or specially embedded transmission elements in the film, via which the driving force and/or the driving torque can be transmitted. The inserts and/or transmission elements increase the cost of the medical barrier and they must also be placed precisely, which increases the amount of work required prior to a medical and/or surgical procedure.

Based on the prior art, the invention is based in particular on but not limited to the object of advantageously further developing an interface for connecting a drive unit to a medical instrument, in particular with regard to costs and/or handling.

This object is achieved according to the invention by an interface for connecting a drive unit to a medical instrument, as described herein and defined in the claims.

The invention relates to an interface for connecting a drive unit to a medical instrument, comprising:
at least one drive-side connecting element with at least one first coupling surface, and
at least one instrument-side connecting element with at least one second coupling surface,
wherein the two connecting elements can be coupled to one another via the respective coupling surfaces with the interposition of a continuous medical barrier, and
at least one holding device which is designed to hold the connecting elements together in a coupled state.

Such a design makes it possible to provide an advantageously further developed interface. In particular, handling can be simplified and costs can be reduced because a continuous medical barrier is used, which is in particular free of special inserts and/or specially embedded transmission elements for transmitting a driving force and/or a driving torque. This makes it possible to eliminate the need for time-consuming, precise placement of the medical barrier. In addition, the cost of each medical application can be reduced because a single-use medical barrier can be kept as cost-effective as possible. In particular, a standard medical barrier can be used.

The interface can be part of a medical robot system, which can additionally comprise the drive unit for providing at least one driving force and/or at least one driving torque and/or the medical instrument. The robot system can also have at least one robot arm, an operating console, an electronics cabinet, in particular an electronics rack, for example for accommodating additional devices, such as an RF generator, a display unit, a patient bed, a storage unit for various medical instruments for use with the interface and/or other units that appear useful to a person skilled in the art. The continuous medical barrier can also be part of the medical robot system. In an operating state, the interface can be carried by the robot arm and be movable thereby.

The medical instrument can be any medical and/or surgical instrument that appears appropriate to a person skilled in the art, which can preferably be carried by the robot arm and be movable thereby. The medical instrument can be permanently or detachably connected to the instrument-side connecting element or can be part of the instrument-side connecting element. The medical instrument can, for example, comprise a laparoscopic unit, an endoscope, a microscope, an exoscope, a scalpel, a drill, a scraper, a clamp, a pair of forceps, a pair of scissors, a syringe, a catheter and/or other units that appear appropriate to a person skilled in the art, which can be driven by means of the drive unit via the interface.

In the coupled state of the interface, the first coupling surface contacts a first side of the medical barrier and the second coupling surface contacts a second side of the medical barrier opposite the first side. In this case, the coupling surfaces can preferably be designed to be complementary to one another, in particular to enable a positive reception and/or a positive clamping of the medical barrier between the connecting elements. The coupling surfaces can each have a total surface area of at least 50 mm², in particular of at least 100 mm² and for example of at least 250 mm².

The connecting elements, when assembled with their coupling surfaces, can form a body that is at least partially rotationally symmetrical about an axis of rotational symmetry, in particular at least partially circular-cylindrical and/or at least partially frustoconical. In this case, the connecting elements can be assembled at least partially transversely to the axis of rotational symmetry. In some embodiments, the drive-side connecting element can be larger than the instrument-side connecting element. The drive-side connecting element can have a receiving space for receiving the instrument-side connecting element. In the coupled state, edges of the interface may be rounded in order to advantageously avoid damage to the medical barrier.

The continuous medical barrier is free of openings, inserts and/or special embedded transmission elements for transmitting a driving force and/or a driving torque. The continuous medical barrier may have a thickness of at most 1 mm over its entire surface area, in particular of at most 0.5 mm and for example of at most 0.1 mm. The continuous medical barrier is preferably designed to be flexible. Particularly preferably, the continuous medical barrier is designed as a correspondingly processed and/or coated fabric and/or preferably as a film, in particular as a plastics film.

The transmission of the driving force and/or the driving torque through the continuous medical barrier can take place mechanically and/or magnetically. In this case, the drive-side connecting element can have a first magnetic element which magnetically interacts with a second magnetic element of the instrument-side connecting element for transmitting the driving force and/or the driving torque. In this case, corresponding drive elements, in particular magnetic elements, of the interface can be arranged in a first receiving space of the drive-side connecting element and/or a second receiving space of the instrument-side connecting element.

The holding device can be designed to hold the connecting elements together in the coupled state in a force-fitting and/or form-fitting manner. For example, it could have interacting magnetic elements by means of which the connecting elements are held together by a magnetic force. In this case, one of the magnetic elements could be a permanent magnetic element which interacts with another permanent magnetic and/or ferromagnetic element. Alternatively, one or both of the magnetic elements could be designed as an electromagnet. It would also be conceivable that the connecting elements are held together in the coupled state by means of an electrical force.

Preferably, the holding device has at least one mechanical holding element for a mechanical connection of the two connecting elements.

In some embodiments, the holding device can have a clamping unit which is designed to encompass at least one of the connecting elements at least in portions, in particular along a peripheral direction, in the coupled state. This makes it possible to provide a structurally simple connection between the connecting elements. Furthermore, damage to the medical barrier can be avoided. In the coupled state, the clamping unit preferably completely encompasses one of the connecting elements, in particular the drive-side connecting element, along the peripheral direction. In the coupled state, the clamping unit can encompass both connecting elements at least in portions.

In some embodiments, the clamping unit can be designed as an in particular rigid clamp. The clamping unit can at least partially form a cylinder jacket surface. Alternatively, the clamping unit could be designed as an in particular flexible and/or rubber-like holding strap, preferably as a ratchet strap, which could be connected and/or connectable at both of its ends to the instrument-side connecting element, for example via metal rings at the two ends of the holding strap, which can be hooked into corresponding hooks on the instrument-side connecting element. This allows a simple construction to be achieved.

In addition, the clamping unit can be arranged on the instrument-side connecting element, whereby operability from a sterile region can be enabled, in particular during a medical and/or surgical procedure, in particular for exchanging the instrument-side connecting element for another instrument-side connecting element. Furthermore, this can enable advantageous protection of the medical barrier, since all elements of the holding device can be provided on an instrument side of the medical barrier.

In some embodiments of the invention, the clamping unit can be guided in the peripheral direction of the connecting elements so as to be movable by means of a guide unit of the instrument-side connecting element between a release position and a clamping position. This can enable safe operation. In particular, incorrect operation and/or jamming can be avoided. The guide unit can be designed as a guide groove into which the clamping unit at least partially engages. The guide groove could have a T-shaped cross-section into which a guide part of the clamping unit with a suitable cross-section engages in a form-fitting manner. Preferably, the guide unit, in particular the guide groove, extends in the peripheral direction over a large part, in particular over at least 50%, preferably over at least 75% and particularly advantageously over at least 90%, of the instrument-side connecting element.

Advantageously, the holding device, in particular on the instrument-side connecting element, can comprise a locking unit which is designed to reversibly fix the clamping unit in a clamping position, in particular in the aforementioned clamping position. In the clamping position, the clamping unit can therefore be fixed at both of its ends to the instrument-side connecting element, wherein the clamping unit can rest against the drive-side connecting element in an intermediate region and in particular can span over it. This allows an advantageous clamping effect to be achieved. In addition, a secure closure of the clamping unit can be provided.

The locking unit can have at least one latching means, in particular a latching lug, which latches with at least one counter-latching means, in particular a latching recess, of the clamping unit in the clamping position. In the release position, the latching means, in particular the latching lug, can be deflected, in particular pressed down, by the clamping unit. In the coupled state, the locking unit can be operable, in particular detachable, from an instrument side of the medical barrier, so that the interface can be released from a sterile side, in particular for exchanging medical instruments. Alternatively or additionally, the locking unit may comprise at least one toggle lever or another mechanism for fixing the clamping unit that appears appropriate to a person skilled in the art.

The clamping unit can advantageously be designed to clamp the medical barrier between itself and an outer surface of the drive-side connecting element. This allows an advantageous fixation of the connecting elements relative to the medical barrier to be achieved. In the coupled state, the clamping unit can be arranged in portions between two layers of the medical barrier.

In some embodiments of the invention and in particular in certain positions of the clamping unit, an inner surface of the clamping unit can run at least partially parallel to an outer surface of the instrument-side connecting element and/or an outer surface of the drive-side connecting element, whereby secure guidance can be achieved. In addition, a receiving gap for the medical barrier can be constant in its dimensions along the peripheral direction.

Furthermore, the drive-side connecting element and/or the instrument-side connecting element can have a receiving groove in which the clamping unit is received at least in part and preferably completely, whereby an advantageous protection of the clamping unit can be achieved. In addition, fixing of the medical barrier can be improved. The receiving grooves can be arranged adjacent to one another and extend completely around the connecting elements in the peripheral direction. In the coupled state, the medical barrier can be arranged at least partially in the receiving groove of the drive-side connecting element. Edges of the receiving groove of the drive-side connecting element and/or the instrument-side connecting element can be rounded in order to advantageously avoid damage to the medical barrier.

In a method for connecting the interface described above, the following steps are preferably carried out:
at least partially covering the coupling surface and/or the outer surface of the drive-side connecting element with the continuous medical barrier,
coupling the drive-side coupling surface to the instrument-side coupling surface with the medical barrier interposed, and
actuating the holding device, in particular closing the clamping unit.

This can simplify handling and reduce costs, since a continuous medical barrier can be used which is in particular free of special inserts and/or specially embedded transmission elements for transmitting a driving force and/or a driving torque. This makes it possible to eliminate the need for time-consuming, precise placement of the medical barrier. In addition, the cost of each medical application can be reduced because a single-use medical barrier can be kept as cost-effective as possible.

The devices and systems described herein and the method described herein should not be limited to the application and embodiment described above. In particular, they can have a number of individual elements, components and units as well as method steps, which differ from a number mentioned herein, in order to fulfill a function described herein. In addition, for the ranges of values specified in this disclosure, values within the stated limits shall also be deemed to be disclosed and to be usable in any manner.

It is in particular pointed out that all features and properties described with regard to a device, but also procedures, can be analogously transferred to methods and can be used within the meaning of the invention and are considered to be co-disclosed. The same applies in the opposite direction. This means that structural features mentioned in relation to methods, i.e., features relating to the device, can also be taken into account, claimed and also counted as part of the disclosure within the scope of the device claims.

The present invention is described below by way of example with reference to the accompanying figures. The drawings, the description, and the claims contain numerous features in combination. A person skilled in the art will also, expediently, consider the features individually and use them in combination as appropriate in the context of the claims.

In the drawings:

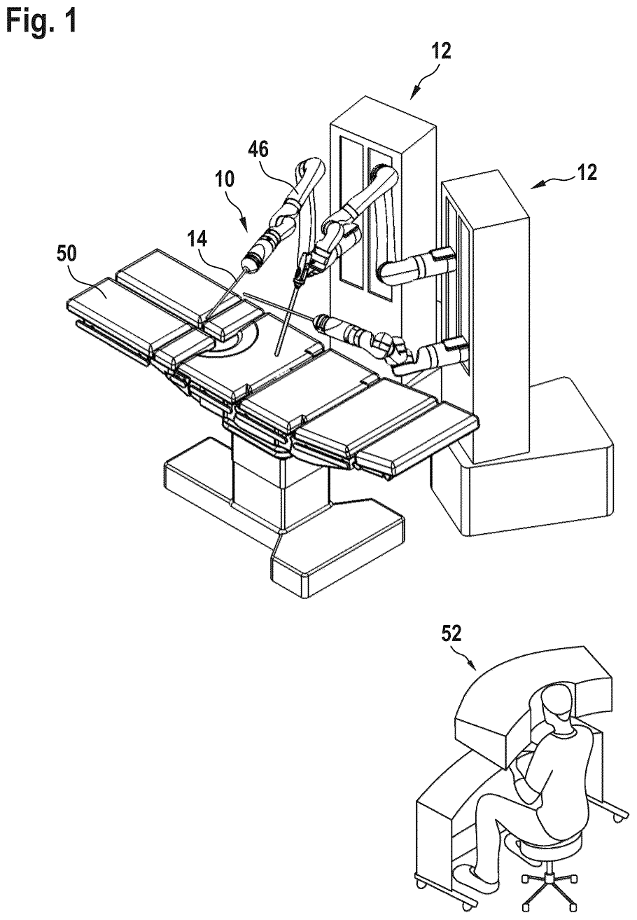

FIG. 1 shows a medical robot system with an interface for connecting a drive unit to a medical instrument,

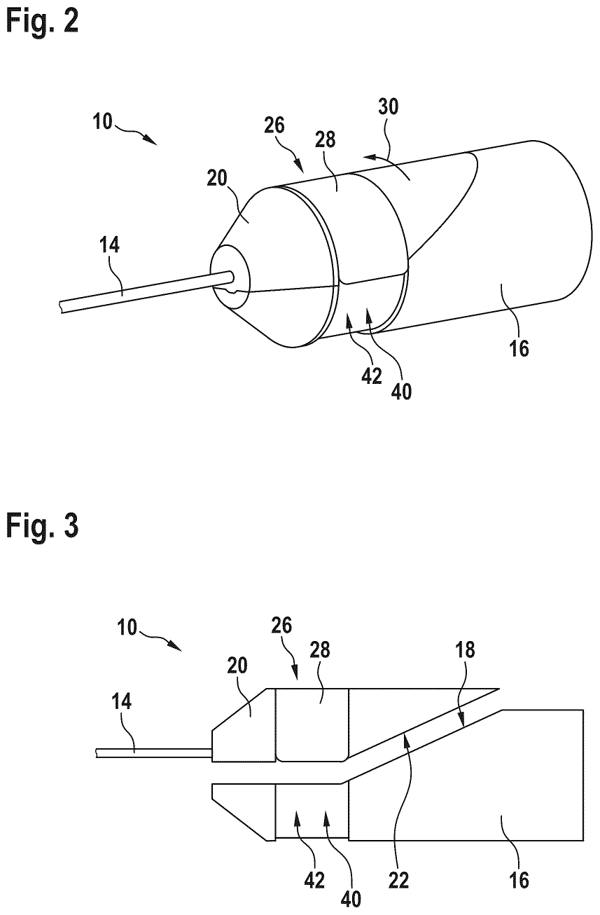

FIG. 2 is a perspective view of the interface with a drive-side connecting element and an instrument-side connecting element in a state applied to one another and with a clamping unit in a release position,

FIG. 3 is a side view of the drive-side connecting element and the instrument-side connecting element in a spatially separated state,

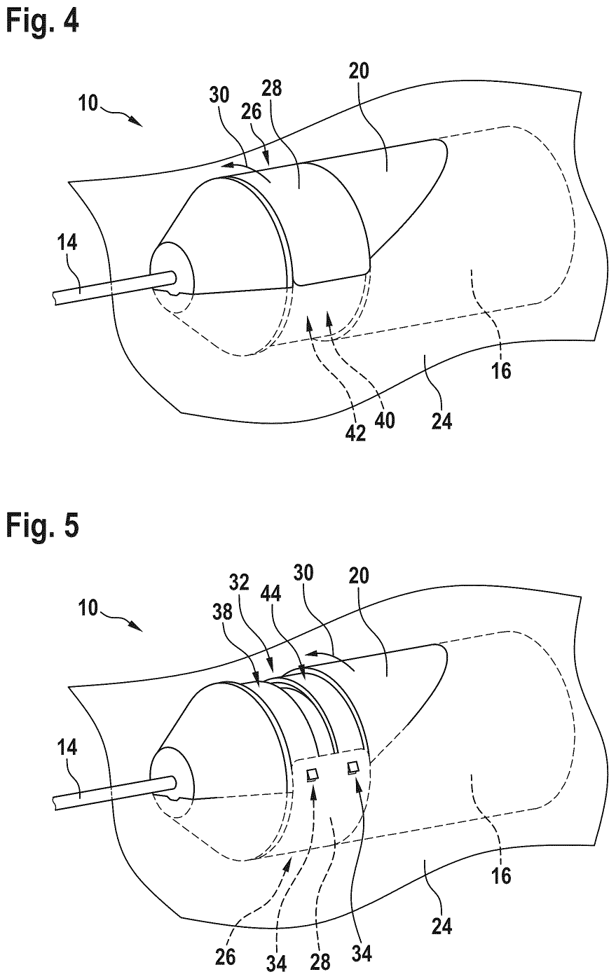

FIG. 4 is a perspective view of the interface with the medical barrier inserted and with the clamping unit in the release position,

FIG. 5 is a perspective view of the interface with the medical barrier inserted and with the clamping unit in the clamping position, and

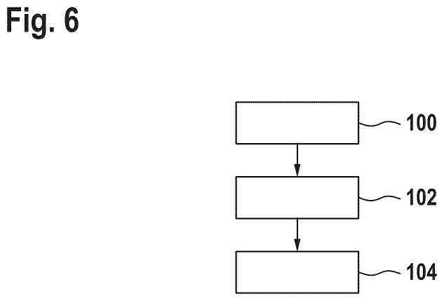

FIG. 6 is a flow chart of a method for connecting the connecting elements with the medical barrier in between.

FIG. 1 shows a medical robot system. The robot system has a patient bed 50. The robot system has a robot arm 46 and a drive unit 12, wherein the robot arm 46 can be controlled via the drive unit 12. The drive unit 12 can be actuated automatically, semi-automatically and/or by medical personnel by means of an operating console 52.

The robot system has a medical instrument 14 which is arranged on the robot arm 46 and can be moved thereby. The medical instrument 14 itself has at least one movable part which is movable relative to another part of the medical instrument 14. The movable part is movable relative to the other part by the drive unit 12. In the present case, the medical instrument 14 is a pair of forceps, although any other instruments deemed appropriate by a person skilled in the art would also be conceivable.

In order to create sterile working conditions, a continuous medical barrier 24 is provided which separates a region around the patient bed 50 from the drive unit 12 and the robot arm 46. A driving force and/or a driving torque for the movable part of the medical instrument 14 is transmitted by means of an interface 10 of the robot system. In a use state, the interface 10 is arranged in the region of the medical barrier 24. In the use state, the interface 10 is held and guided by the robot arm 46.

The interface 10 has a drive-side connecting element 16 and an instrument-side connecting element 20, which are shown in FIG. 2 in a perspective view in a state applied to one another. FIG. 3 shows the connecting elements 16, 20 in a spatially separated state. The medical instrument 14 is rigidly connected to the instrument-side connecting element 20. Alternatively, a detachable connection of the medical instrument 14 to the instrument-side connecting element 20 would also be conceivable.

The drive-side connecting element 16 has a first coupling surface 18. The instrument-side connecting element 20 comprises a second coupling surface 22. The coupling surfaces 18, 22 are complementary to each other and allow a positive connection of the connecting elements 16, 20, as shown in FIG. 2.

The connecting elements 16, 20, when assembled, form a body that is at least partially circular-cylindrical and at least partially frustoconical. The drive-side connecting element 16 is larger than the instrument-side connecting element 20 and has a receiving space for receiving the instrument-side connecting element 20 (see FIG. 3).

The coupling surfaces 18, 22 are designed to clamp the medical barrier 24 (see FIGS. 4 and 5). The connecting elements 16, 20 can be coupled to one another via their respective coupling surfaces 18, 22 with the interposition of the medical barrier 24. The medical barrier 24 is a continuous plastics film which is in particular free of openings. In the use state, the two connecting elements 16, 20 are separated from each other by the medical barrier 24. In this case, in the use state, the instrument-side connecting element 20 together with the medical instrument 14 is located on a sterile side of the medical barrier 24.

A transmission of a driving force and/or a driving torque through the continuous medical barrier 24 occurs for example magnetically (not shown). In this case, the drive-side connecting element 16 has a first magnetic element in its interior, which magnetically interacts with a second magnetic element of the instrument-side connecting element 20 for transmitting the driving force and/or the driving torque. In this case, the first magnetic element can be driven by the drive unit 12.

The interface 10 has a holding device 26 which is designed to hold the connecting elements 16, 20 together in a coupled state and in particular in the use state.

The holding device 26 has a clamping unit 28 which is designed to encompass at least one of the connecting elements 16, 20 at least in portions in the coupled state and in particular in the use state. In this case, the clamping unit 28 is designed to completely encompass the drive-side connecting element 16 in the peripheral direction 30 of the connecting elements 16, 20 in the coupled state and in particular in the use state. The clamping unit 28 is designed as a clamp and at least partially forms a cylinder jacket surface.

The clamping unit 28 is arranged on the instrument-side connecting element 20. The clamping unit 28 is movably guided in the peripheral direction 30 by means of a guide unit 32 (cf. FIG. 5) of the instrument-side connecting element 20 between a release position according to FIG. 4 and a clamping position according to FIG. 5. When the clamping unit 28 is transferred from the release position to the clamping position, the clamping unit 28 is to be moved along the peripheral direction 30, specifically in the direction of the arrow shown in FIG. 2, around the connecting elements 16, 20. In this case, an inner surface of the clamping unit 28 runs at least partially parallel to an outer surface 38 of the instrument-side connecting element 20 and an outer surface 40 of the drive-side connecting element 16.

The guide unit 32 is designed as a guide groove into which the clamping unit 28 at least partially engages. The guide groove has a T-shaped cross-section into which a guide part of the clamping unit 28 with a matching cross-section engages in a form-fitting manner (not shown). The guide groove encompasses almost the entire instrument-side connecting element 20, in the peripheral direction.

The holding device 26 comprises a locking unit 34 which is designed to reversibly fix the clamping unit 28 in the clamping position. In the clamping position, the clamping unit 28 is fixed at both ends to the instrument-side connecting element 20, wherein the clamping unit 18 rests against the drive-side connecting element 16 in an intermediate region and spans it. The locking unit 34 has two latching lugs which, in the clamping position, are latched with two latching recesses 48 of the clamping unit 28 (see FIG. 5). In the release position, the latching lugs are pressed down by the clamping unit 28 (not shown).

The drive-side connecting element 16 has a receiving groove 42 (see FIGS. 2, 3 and 4). The instrument-side connecting element 20 comprises a receiving groove 44 (see FIG. 5). The receiving grooves 42, 44 are arranged adjacent to one another and extend completely around the connecting elements 16, 20, in the peripheral direction 30. The clamping unit 28 is received flush in the receiving grooves 42, 44 and can be moved along these. The guide unit 32 is arranged in the receiving groove 44.

The clamping unit 28 is designed to clamp the medical barrier 24, as shown in FIG. 5, between itself and the outer surface 40 of the drive-side connecting element 16.

FIG. 6 shows a flow chart of a method for connecting the connecting elements 16, 20 with the medical barrier 24 in between. In a step 100, the first coupling surface 18 and the outer surface 40 of the drive-side connecting element 16 are at least partially covered with the medical barrier 24. In a step 102, the instrument-side connecting element 20 is connected to the first coupling surface 18 via its second coupling surface 22 with the medical barrier 24 in between. Finally, in a step 104, the clamping unit 28 is actuated.

### LIST OF REFERENCE SIGNS

10 interface
12 drive unit
14 medical instrument
16 drive-side connecting element
18 first coupling surface
20 instrument-side connecting element
22 second coupling surface
24 medical barrier
26 holding device
28 clamping unit
30 peripheral direction
32 guide unit
34 locking unit
38 outer surface
40 outer surface
42 receiving groove
44 receiving groove
46 robot arm
48 latching recess
50 patient bed
52 operating console
100 step
102 step
104 step

1. Interface (10) for connecting a drive unit (12) to a medical instrument (14), comprising:
at least one drive-side connecting element (16) with at least one first coupling surface (18), and
at least one instrument-side connecting element (20) with at least one second coupling surface (22),
wherein the two connecting elements (16, 20) can be coupled to one another via the respective coupling surfaces (18, 22) with the interposition of a continuous medical barrier (24), and
at least one holding device (26) which is designed to hold the connecting elements (16, 20) together in a coupled state.

2. Interface (10) according to claim 1, wherein the holding device (26) has a clamping unit (28) which is designed to at least partially encompass at least one of the connecting elements (16, 20) in the coupled state.

3. Interface (10) according to claim 2, wherein the clamping unit (28) is arranged on the instrument-side connecting element (20).

4. Interface (10) according to either claim 2 or claim 3, wherein the clamping unit (28) is movably guided in the peripheral direction (30) of the connecting elements (16, 20) by means of a guide unit (32) of the instrument-side connecting element (20) between a release position and a clamping position.

5. Interface (10) according to any of claims 2 to 4, wherein the holding device (26) comprises a locking unit (34) which is designed to reversibly fix the clamping unit (28) in a clamping position.

6. Interface (10) according to any of claims 2 to 5, wherein an inner surface of the clamping unit (28) runs at least partially parallel to an outer surface (38) of the instrument-side connecting element (20) and/or an outer surface (40) of the drive-side connecting element (16).

7. Interface (10) according to any of claims 2 to 6, wherein the drive-side connecting element (16) and/or the instrument-side connecting element (20) has a receiving groove (42, 44) in which the clamping unit (28) is at least partially received.

8. Interface (10) according to any of claims 2 to 7, wherein the clamping unit (28) is designed to clamp the medical barrier (24) between itself and an outer surface (40) of the drive-side connecting element (16).

9. Medical robot system comprising:
an interface (10) according to any of the preceding claims.

10. Medical robot system according to claim 9, further comprising:
the drive unit (12), which is connected to the drive-side connecting element (16).

11. Medical robot system (10) according to either claim 9 or claim 10, further comprising:
the medical instrument (14) which is connected to the instrument-side connecting element (20).

12. Medical robot system according to any of claims 9 to 11, further comprising:
the medical barrier (24) which is clamped between the drive-side connecting element (16) and the instrument-side connecting element (20).
